(19) United States
(12) Patent Application Publication
(10) Pub. No.: US 2026/0367038 A1
(43) Pub. Date: Nov. 19, 2026

(54) METHOD FOR ANALYZING NITROGEN ISOTOPES IN LUNAR SOIL

(71) Applicant: Institute of Geology and Geophysics,CAS, Beijing (CN)

(72) Inventors: Ziheng LIU, Beijing (CN); Huaiyu HE, Beijing (CN); Jiannan LI, Beijing (CN); Fei SU, Beijing (CN)

(21) Appl. No.: 19/305,263

(22) Filed: Aug. 20, 2025

(30) Foreign Application Priority Data
May 14, 2025 (CN) 202510622076.6

(51) Int. Cl. G01N 27/626; G01N 1/44; G01N 33/00; H01J 49/04

(52) U.S. Cl. CPC G01N 27/626; G01N 1/44; G01N 33/0036; H01J 49/0422; H01J 49/0468

(57) ABSTRACT

A method for analyzing nitrogen isotopes in lunar soil includes: fabricating an analysis system including a heating chamber and a quadrupole mass spectrometer, where a connecting pipe is provided with a control valve; preparing multiple gas mixtures of carbon monoxide and nitrogen with different ratios, measuring by an analysis system, recording a slope of curve of a linear variation of a gas pressure over time to establish a database; placing the lunar soil in the heating chamber for heating, measuring released gases, recording a slope, determining a mixing ratio according to the measured slope and the database, and subtracting an amount of carbon monoxide to obtain a nitrogen isotope ratio.

### CROSS-REFERENCE TO RELATED APPLICATION

This patent application claims the benefit and priority of Chinese Patent Application No. 202510622076.6 filed with the China National Intellectual Property Administration on May 14, 2025, the disclosure of which is incorporated by reference herein in its entirety as part of the present application.

### Technical Field

The present disclosure relates to the technical field of lunar soil analysis, and in particular, to a method for analyzing nitrogen isotopes in lunar soil.

### BACKGROUND

In the field of celestial science research, a deep research into lunar materials is crucial to reveal the formation and evolution mechanism of the moon. The analysis of nitrogen isotopes plays a unique and key role in this research.

Nitrogen isotope analysis can help researchers determine the source of lunar materials and deeply understand the evolution of volatile elements after the formation of the moon. Through careful analysis of the distribution of nitrogen isotopes in lunar soil and rocks, the migration law of volatile substances in the moon as well as the cycle and variation of nitrogen elements in long-term evolution of the moon can be clearly understood. In addition, analysis of a proportion of nitrogen isotopes in the lunar soil may also be used to research the composition of nitrogen in solar wind and its dynamic variation with time, indirectly tracing evolution history of the solar wind.

In addition, the source of nitrogen on the lunar surface is rather complex, which is not only from the solar wind, but also from the impact of extraterrestrial materials such as comets and asteroids. Composition characteristics of the nitrogen isotopes are like "fingerprints", which can effectively distinguish these materials from different sources, thereby providing important clues for further research on the input of the extraterrestrial materials. Moreover, by comparing the composition of nitrogen isotopes of the lunar surface with that of planets such as Earth and Mars, it is helpful for researchers to establish a more accurate planetary system material exchange model.

At present, nitrogen has two stable isotopes, namely, nitrogen-14 and nitrogen-15. Composition information of nitrogen isotopes in nitrogen can be determined by analyzing a ratio of nitrogen with mass-to-charge ratios of 28 and 29. However, the actual measurement process is faced with a severe technical problem: a mass number of carbon monoxide released by heating is the same as that of nitrogen, which is 28. In mass spectrometer analysis, the carbon monoxide interferes with a position of a nitrogen peak, resulting in deviation of a measurement result, and the composition information of the nitrogen isotopes cannot be accurately obtained, which seriously affects the accuracy and reliability of nitrogen isotope analysis of the lunar soil, and hinders the in-depth development of related research.

Therefore, it is urgent to develop a method that can effectively avoid interference of carbon monoxide and accurately analyze nitrogen isotopes in lunar soil, thereby promoting a further development of lunar science research and planetary science related fields.

### SUMMARY

The present disclosure aims to provide a method for analyzing nitrogen isotopes in lunar soil, so as to solve the problems in the prior art, thereby obtaining a relative ratio of nitrogen-14 to nitrogen-15 in a gas released from the lunar soil.

To achieve the objective above, the present disclosure employs the following technical solution:
the present disclosure provides a method for analyzing nitrogen isotopes in lunar soil, including the following steps:
   S1, fabricating an analysis system, wherein the analysis system comprises a heating chamber and a quadrupole mass spectrometer, a gas outlet of the heating chamber is in communication with an air inlet of the quadrupole mass spectrometer via a connecting pipe, the connecting pipe is provided with a control valve configured to control opening and closing of the connecting pipe;
   S2, preparing a plurality of gas mixtures, wherein each of the gas mixtures comprises carbon monoxide and nitrogen, mixing ratios of carbon monoxide to nitrogen in different gas mixtures are different; measuring the gas mixtures in sequence by the analysis system, wherein during measurement, each of the gas mixtures is introduced into the heating chamber, and then into the quadrupole mass spectrometer via the connecting pipe, an ion with a mass-to-charge ratio of 28 is selected by the quadrupole mass spectrometer, and a slope of curve of a linear variation of a gas pressure over time in the quadrupole mass spectrometer when each of the gas mixtures is measured is recorded and is marked as a calibration slope; establishing a database according to a mixing ratio of the carbon monoxide to the nitrogen in each of the gas mixtures and a corresponding calibration slope, and drawing a graph of an variation of the calibration slope over the mixing ratio; and
   S3, placing lunar soil to be analyzed into the heating chamber for heating to release gases from the lunar soil, detecting a strength of an ion with a mass-to-charge ratio of 28 and a strength of an ion with a mass-to-charge ratio of 29 in the gases released from the lunar soil by the quadrupole mass spectrometer, and recording a slope of curve of an variation of a gas pressure over time in the quadrupole mass spectrometer when detecting the strength of the ion with the mass-to-charge ratio of 28 in the gases released from the lunar soil as a measured slope; then, calculating a relative ratio of the ion with the mass-to-charge ratio of 28 to the ion with the mass-to-charge ratio of 29, and analyzing and obtaining a mixing ratio of carbon monoxide to nitrogen in the gases released from the lunar soil according to the measured slope, the database and the graph; and then, on the basis of the relative ratio, subtracting an amount of the carbon monoxide from the amount of the ion with the mass-to-charge ratio of 28, and calculating a ratio of residual ion with the mass-to-charge ratio of 28 to the ion with the mass-to-charge ratio of 29, which is a relative ratio of nitrogen-14 to nitrogen-15 in the gases released from the lunar soil.

Preferably, the heating chamber includes a housing and a heating element attached to an outer wall of the housing, and the housing is sealed and is provided with a movable door.

Preferably, the heating element includes an electric heating plate.

Preferably, the housing is made of a titanium alloy.

Preferably, the analysis system further includes a first storage tank and a second storage tank, the first storage tank is in communication with the housing via a first pipeline, and the second storage tank is in communication with the housing via a second pipeline; the first pipeline is provided with a first gas pump and a first gas flowmeter, the first gas flowmeter is closer to the housing than the first gas pump, the second pipeline is provided with a second gas pump and a second gas flowmeter, and the second gas flowmeter is closer to the housing than the second gas pump.

Preferably, the analysis system further includes a control unit, the first gas flowmeter and the second gas flowmeter are in signal connection with the control unit, the control unit is able to turn off the first gas pump when a detection value of the first gas flowmeter reaches a set value, and the control unit is able to turn off the second gas pump when a detection value of the second gas flowmeter reaches a set value.

Preferably, the control unit includes an field programmable gate array (FPGA).

Preferably, the first pipeline is further provided with a first valve and a second valve, the first valve is located between the first storage tank and the first gas pump, the second valve is located between the first gas flowmeter and the housing; the second pipeline is further provided with a third valve and a fourth valve, the third valve is located between the second storage tank and the second gas pump, and the fourth valve is located between the second gas flowmeter and the housing.

Preferably, the analysis system further includes a first vacuum pump and a second vacuum pump; the heating chamber is in communication with an air suction port of the first vacuum pump via a third pipeline, the third pipeline is provided with a fifth valve, and an air outlet of the quadrupole mass spectrometer is in communication with an air suction port of the second vacuum pump via a fourth pipeline.

Preferably, each of the first valve, the second valve, the third valve, the fourth valve, the fifth valve and the control valve is an electromagnetic valve.

Compared with the prior art, the present disclosure has the following technical effects:

In the prior art, as a mass number of carbon monoxide released by heating is the same as that of nitrogen, which is 28, a position of a nitrogen peak in a mass spectrometer will be disturbed, which seriously affects the accuracy of nitrogen isotope analysis in lunar soil. In the present disclosure, the slope of curve of linear variation of the gas pressure over time in the quadrupole mass spectrometer is calibrated by means of the gas mixtures of carbon monoxide and nitrogen with different mixing ratios and a database is established. When analyzing the gases released from lunar soil, the mixing ratio is determined according to the measured slope and the database, and then the amount of the carbon monoxide is subtracted, so as to effectively avoid the interference of the carbon monoxide on nitrogen isotope measurement, significantly improving the accuracy and reliability of a result of nitrogen isotope analysis of the lunar soil, and providing more accurate data support for related research.

Furthermore, in the present disclosure, the method for analyzing nitrogen isotopes in lunar soil only needs to construct an analysis system composed of a heating chamber and a quadrupole mass spectrometer, and opening and closing of the gas is achieved by means a control valve. During measurement, slope data is obtained by means of a linear variation of a gas pressure over time, the operation process is relatively simple and clear. Neither does it need a complex chemical reaction to convert the carbon monoxide into other gases, not does it need to use physical removal means such as molecular sieve or membrane filtration, nor does it need to improve a mass analysis range of a mass spectrometer, such that the increase of the weight of the on-orbit measurement payload due to the use of these methods above is avoided, experimental cost and operation difficulty are reduced, and the method has high feasibility and practicability in practical application.

Furthermore, in the present disclosure, the method for analyzing nitrogen isotopes in lunar soil solves the problem of measurement interference in the prior art, an accurate nitrogen isotope ratio in the lunar soil can be obtained, and reliable technical means are provided for these researches. This method is helpful for promoting the further in-depth development of lunar scientific research and related fields of planetary science, and has important scientific value and research significance.

### BRIEF DESCRIPTION OF THE DRAWINGS

To describe the technical solutions of embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a structural diagram of an analysis system in a method for analyzing nitrogen isotopes in lunar soil according to the present disclosure;

FIG. 2 is a structural diagram of a heating chamber in an analysis system according to the present disclosure; and

FIG. 3 is a graph showing a variation of a gas pressure over time in a chamber of a quadrupole mass spectrometer.

In the drawings: 1-heating chamber; 101-housing; 102-heating element; 103-movable door; 2-quadrupole mass spectrometer; 3-first storage tank; 4-first gas pump; 5-first gas flowmeter; 6-second storage tank; 7-second gas pump; 8-second gas flowmeter; 9-first vacuum pump; 10-second vacuum pump; 11-first valve; 12-second valve; 13-third valve; 14-fourth valve; 15-fifth valve; 16-control valve; 17-connecting pipe; 18-first pipeline; 19-second pipeline; 20-third pipeline; and 21-fourth pipeline.

### DETAILED DESCRIPTION OF THE EMBODIMENTS

The following clearly and completely describes the technical solutions in embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

The present disclosure aims to provide a method for analyzing nitrogen isotopes in lunar soil, so as to solve the problems in the prior art, thereby obtaining a relative ratio of nitrogen-14 to nitrogen-15 in a gas released from the lunar soil.

To make the objectives, features and advantages of the present disclosure more clearly, the present disclosure is further described in detail below with reference to the accompanying drawings and specific implementations.

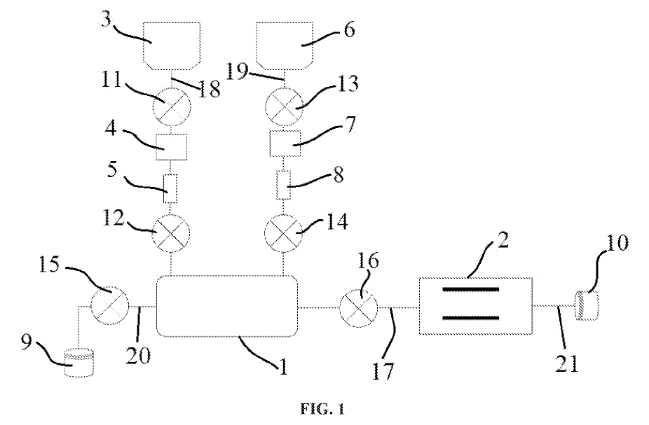

As shown in FIGS. 1-FIG. 3, the embodiment provides a method for analyzing nitrogen isotopes in lunar soil, including the following steps.

S1, an analysis system is fabricated. The analysis system includes a heating chamber 1 and a quadrupole mass spectrometer 2, a gas outlet of the heating chamber is in communication with a gas inlet of the quadrupole mass spectrometer 2 via a connecting pipe 17, the connecting pipe 17 is provided with a control valve 16, and the control valve 16 is configured to control the opening and closing of the connecting pipe 17.

S2, multiple gas mixtures are prepared. Each gas mixture includes carbon monoxide and nitrogen, mixing ratios of carbon monoxide to nitrogen in different gas mixtures are different. The gas mixtures then are measured in sequence by the analysis system. During measurement, the gas mixture is introduced into the heating chamber 1, and then into the quadrupole mass spectrometer 2 via the connecting pipe 17. An ion with a mass-to-charge ratio of 28 is selected by the quadrupole mass spectrometer 2, and when each gas mixture is measured, a slope of curve of a linear variation of a gas pressure over time in the quadrupole mass spectrometer 2 is recorded and marked as a calibration slope. A database is established according to the mixing ratio of the carbon monoxide to the nitrogen in each gas mixture and a corresponding calibration slope, and a graph of an variation of the calibration slope over the mixing ratio is drawn.

S3. the lunar soil to be analyzed is placed in the heating chamber 1 for heating to release gases from the lunar soil, a strength of an ion with a mass-to-charge ratio of 28 and a strength of an ion with a mass-to-charge ratio of 29 in the gases released from the lunar soil are detected by the quadrupole mass spectrometer 2, and when detecting the strength of the ion with the mass-to-charge ratio of 28, the slope of curve a linear variation of a gas pressure over time in the quadrupole mass spectrometer 2 is recorded as a measured slope. Then, a relative ratio of the ion with the mass-to-charge ratio of 28 to the ion with the mass-to-charge ratio of 29 is calculated. According to the measured slope, the database and the graph, the mixing ratio of the carbon monoxide to the nitrogen in the gases released from the lunar soil is analyzed and obtained. Then, According to the relative ratio calculated, an amount of carbon monoxide is subtracted from the amount of ion with the mass-to-charge ratio of 28, and a ratio of residual ion with the mass-to-charge ratio of 28 to the ion with the mass-to-charge ratio of 29 is calculated, that is, a relative ratio of nitrogen-14 to nitrogen-15 in the gases released from the lunar soil is calculated.

A phenomenon that the gas pressure of the gas mixture varies linearly with time in the process of being measured by the quadrupole mass spectrometer 2 is specifically described as follows.

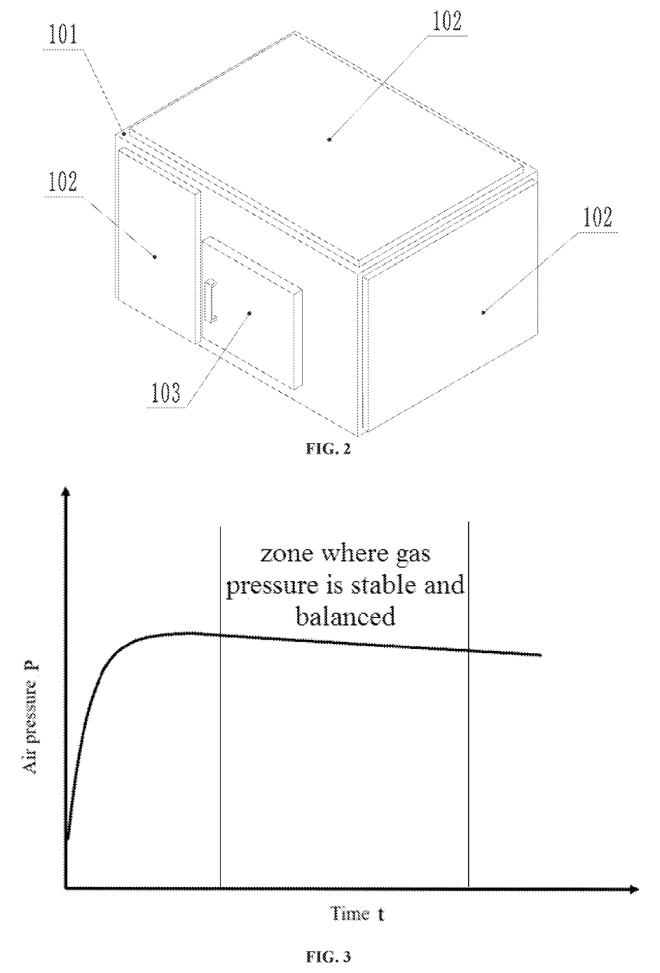

It is assumed that a partial pressure of a gas to be measured in the heating chamber 1 is P1, a control valve 16 is opened to introduce the gas into the quadrupole mass spectrometer 2 via the connecting pipe 17, and the partial pressure of the gas in a chamber of the quadrupole mass spectrometer chamber is defined as P2. The intake conductance of the quadrupole mass spectrometer 2 is defined as U1, and the exhaust conductance of the quadrupole mass spectrometer 2 is defined as U2, and it is assumed that a lunar surface environment is an absolute vacuum. As an exhaust volume is smaller than an intake volume in the process of gas pressure balance between the intake and the exhaust (that is, in the balance process where the intake volume is approximately equal to the exhaust volume), it is assumed that the partial pressure P1 before intake remains unchanged, then after the intake and the exhaust are balanced, the following relationship between them is satisfied: P1* U1 = P2 * U2. In fact, the gas pressure in the chamber of the quadrupole mass spectrometer increases rapidly, and then decreases gradually with ionization and exhaust. After the gas is stable and balanced, the gas pressure basically decreases linearly, and a variation of the gas pressure over time is shown in FIG. 3.

However, a slope of curve of an variation of a gas pressure over time after balance is mainly affected by differences in a gas exhaust rate and ionization efficiency. This variation of the gas pressure enables the quadrupole mass spectrometer 2 to measure the ions with the mass-to-charge ratios of 28 and 29 during the whole variation cycle of the gas pressure. After the calibration slopes corresponding to gas mixtures with different mixing ratios of carbon monoxide to nitrogen are measured and the database is established, a graph of an variation of the calibration slope over the mixing ratio is drawn. In the process of measuring the gas mixture of nitrogen and carbon monoxide with an unknown ratio, a slope of a curve of an variation of the peak height of the ions with the mass-to-charge ratio of 28 after stabilization can reflect the mixing ratio of the nitrogen to the carbon monoxide in the gas mixture. Then, according to the mixing ratio of the nitrogen to the carbon monoxide, an amount of the carbon monoxide is subtracted from a total amount of the ion with the mass-to-charge ratio of 28 and a relative ratio of nitrogen-14 to nitrogen-15 in the gas released from the lunar soil can be obtained.

In this embodiment, the slope of curve of linear variation of the gas pressure over time in the quadrupole mass spectrometer 2 is calibrated by means of the gas mixtures of carbon monoxide and nitrogen with different mixing ratios, a database is established, and a graph of an variation of the calibration slope over the mixing ratio is drawn. When analyzing the gases released from lunar soil, the mixing ratio is determined according to the measured slope and the database, and then the amount of the carbon monoxide is subtracted, so as to effectively avoid the interference of the carbon monoxide on nitrogen isotope measurement, significantly improving the accuracy and reliability of a result of nitrogen isotope analysis of the lunar soil, and providing more accurate data support for related research.

The heating chamber 1 is an accommodating chamber that can be used to heat a substance in the chamber. In this embodiment, as shown in FIG. 2, the heating chamber 1 includes a housing 101 and heating elements 102 attached to an outer wall of the housing 101. The housing 101 is sealed, and the housing 101 is provided with a movable door 103. The movable door 103 is disposed to facilitate the addition of the lunar soil into the heating chamber 1. It is easy to understand that the movable door 103 can be opened and closed. It should be noted that to improve the heating effect, the heating element 102 can cover all the outer wall of the housing 101 and the outer wall of the movable door 103. In practical application, technicians can adaptively adjust mounting positions and number of the heating elements 102 according to actual needs.

In an alternative of this embodiment, the heating element 102 includes an electric heating plate, which is attached to the outer wall of the housing 101. The electric heating plate can rapidly heat the housing 101, and the temperature distribution of the electric heating plate is uniform, which can improve heating uniformity of the housing 101.

In an alternative of the present disclosure, the housing 101 is made of a titanium alloy. The titanium alloy has high strength, which can bear a larger external force and pressure, and ensures that the housing 101 is structurally stable and not easily deformed in various complicated working environments. Meanwhile, the density of the titanium alloy is relatively small, which is much lighter than many conventional metal materials such as steel. For some equipment or products that have requirement for weight, the titanium alloy is conducive to reducing the overall weight, improving transportation efficiency, reducing energy consumption and enhancing the portability of the equipment.

In an alternative of this embodiment, preferably, the analysis system further includes a first storage tank 3 and a second storage tank 6. The first storage tank 3 is in communication with the housing 101 via a first pipeline 18, and the second storage tank 6 is in communication with the housing 101 via a second pipeline 19. The first storage tank 3 is configured for storing carbon monoxide, and the second storage tank 6 is configured for storing nitrogen. The first pipeline 18 is provided with a first gas pump 4 and a first gas flowmeter 5, the first gas flowmeter 5 is closer to the housing 101 than the first gas pump 4, and carbon monoxide gas can be quantitatively conveyed through the cooperation of the first gas pump 4 and the first gas flowmeter 5. The second pipeline 19 is provided with a second gas pump 7 and a second gas flowmeter 8, the second gas flowmeter 8 is closer to the housing 101 than the second gas pump 7, and nitrogen can be quantitatively conveyed into the heating chamber 1 through the cooperation of the second gas flowmeter 8 and the second gas pump 7, thereby controlling the mixing ratio of the carbon monoxide to the nitrogen conveniently.

In an alternative of this embodiment, preferably, the analysis system further includes a control unit, the first gas flowmeter 5 and the second gas flowmeter 8 are in signal connection with the control unit, the control unit can turn off the first gas pump 4 when a detection value of the first gas flowmeter 5 reaches a set value, and the control unit can turn off the second gas pump 7 when a detection value of the second gas flowmeter 8 reaches a set value. By setting set values of carbon monoxide and nitrogen in the control unit, the corresponding gas pump can be automatically turned off after conveying a set amount of carbon monoxide and nitrogen, so that the automation of the system is improved.

In an alternative of this embodiment, the control unit includes an FPGA. FPGA is an existing programmable logic chip, an operating principle of which is not described in detail in this embodiment.

In an alternative of this embodiment, preferably, the first pipeline 18 is further provided with a first valve 11 and a second valve 12, the first valve 11 is located between the first storage tank 3 and the first gas pump 4, the second valve 12 is located between the first gas flowmeter 5 and the housing 101. The second pipeline 19 is further provided with a third valve 13 and a fourth valve 14, the third valve 13 is located between the second storage tank 6 and the second gas pump 7, and the fourth valve 14 is located between the second gas flowmeter 8 and the housing 101.

In an alternative of this embodiment, preferably, the analysis system further includes a first vacuum pump 9 and a second vacuum pump 10. The heating chamber 1 is in communication with an air suction port of the first vacuum pump 9 via a third pipeline 20, and the third pipeline 20 is provided with a fifth valve 15. A gas outlet of the quadrupole mass spectrometer 2 is in communication with an air suction port of the second vacuum pump 10 via a fourth pipeline 21. The reason to dispose the first vacuum pump 9 is that the heating chamber 1 can be vacuumized by the first vacuum pump 9 before the gas mixture is introduced into the heating chamber 1, thereby preventing an original gas in the heating chamber 1 from being mixed with the gas mixture to affect the detection result. Similarly, before the quadrupole mass spectrometer 2 is used for detection, the chamber of the quadrupole mass spectrometer 2 can be vacuumized by the second vacuum pump 10 to prevent an original gas in the chamber of the quadrupole mass spectrometer 2 from affecting the detection result.

In an alternative of this embodiment, preferably, each of the first valve 11, the second valve 12, the third valve 13, the fourth valve 14, the fifth valve 15 and the control valve 16 is an electromagnetic valve. The electromagnetic valve is rapid in response speed, and thus can be opened and closed within short time.

Specific examples are used herein for illustration of the principles and implementations of the present disclosure. The description of the embodiments is merely used to help illustrate the method and its core principles of the present disclosure. In addition, those of ordinary skill in the art can make various modifications in terms of specific implementations and scope of application in accordance with the teachings of the present disclosure. In conclusion, the content of this specification shall not be construed as a limitation to the present disclosure.

What is claimed is:

1. A method for analyzing nitrogen isotopes in lunar soil, comprising:
S1, fabricating an analysis system, wherein the analysis system comprises a heating chamber and a quadrupole mass spectrometer, a gas outlet of the heating chamber is in communication with an air inlet of the quadrupole mass spectrometer via a connecting pipe, the connecting pipe is provided with a control valve configured to control opening and closing of the connecting pipe;
S2, preparing a plurality of gas mixtures, wherein each of the gas mixtures comprises carbon monoxide and nitrogen, mixing ratios of carbon monoxide to nitrogen in different gas mixtures are different; measuring the gas mixtures in sequence by the analysis system, wherein during measurement, each of the gas mixtures is introduced into the heating chamber, and then into the quadrupole mass spectrometer via the connecting pipe, an ion with a mass-to-charge ratio of 28 is selected by the quadrupole mass spectrometer, and a slope of curve of a linear variation of a gas pressure over time in the quadrupole mass spectrometer when each of the gas mixtures is measured is recorded and is marked as a calibration slope; establishing a database according to a mixing ratio of the carbon monoxide to the nitrogen in each of the gas mixtures and a corresponding calibration slope, and drawing a graph of an variation of the calibration slope over the mixing ratio; and
S3, placing lunar soil to be analyzed into the heating chamber for heating to release gases from the lunar soil, detecting a strength of an ion with a mass-to-charge ratio of 28 and a strength of an ion with a mass-to-charge ratio of 29 in the gases released from the lunar soil by the quadrupole mass spectrometer, and recording a slope of curve of an variation of a gas pressure over time in the quadrupole mass spectrometer when detecting the strength of the ion with the mass-to-charge ratio of 28 in the gases released from the lunar soil as a measured slope; then, calculating a relative ratio of the ion with the mass-to-charge ratio of 28 to the ion with the mass-to-charge ratio of 29, and analyzing and obtaining a mixing ratio of carbon monoxide to nitrogen in the gases released from the lunar soil according to the measured slope, the database and the graph; and then, on the basis of the relative ratio, subtracting an amount of the carbon monoxide from the amount of the ion with the mass-to-charge ratio of 28, and calculating a ratio of residual ion with the mass-to-charge ratio of 28 to the ion with the mass-to-charge ratio of 29, which is a relative ratio of nitrogen-14 to nitrogen-15 in the gases released from the lunar soil.

2. The method for analyzing nitrogen isotopes in lunar soil according to claim 1, wherein the heating chamber comprises a housing and a heating element attached to an outer wall of the housing, and the housing is sealed and is provided with a movable door.

3. The method for analyzing nitrogen isotopes in lunar soil according to claim 2, wherein the heating element comprises an electric heating plate.

4. The method for analyzing nitrogen isotopes in lunar soil according to claim 2, wherein the housing is made of a titanium alloy.

5. The method for analyzing nitrogen isotopes in lunar soil according to claim 2, wherein the analysis system further comprises a first storage tank and a second storage tank, the first storage tank is in communication with the housing via a first pipeline, and the second storage tank is in communication with the housing via a second pipeline; the first pipeline is provided with a first gas pump and a first gas flowmeter, the first gas flowmeter is closer to the housing than the first gas pump, the second pipeline is provided with a second gas pump and a second gas flowmeter, and the second gas flowmeter is closer to the housing than the second gas pump.

6. The method for analyzing nitrogen isotopes in lunar soil according to claim 5, wherein the analysis system further comprises a control unit, the first gas flowmeter and the second gas flowmeter are in signal connection with the control unit, the control unit is able to turn off the first gas pump when a detection value of the first gas flowmeter reaches a set value, and the control unit is able to turn off the second gas pump when a detection value of the second gas flowmeter reaches a set value.

7. The method for analyzing nitrogen isotopes in lunar soil according to claim 6, wherein the control unit comprises an field programmable gate array (FPGA).

8. The method for analyzing nitrogen isotopes in lunar soil according to claim 5, wherein the first pipeline is further provided with a first valve and a second valve, the first valve is located between the first storage tank and the first gas pump, the second valve is located between the first gas flowmeter and the housing; the second pipeline is further provided with a third valve and a fourth valve, the third valve is located between the second storage tank and the second gas pump, and the fourth valve is located between the second gas flowmeter and the housing.

9. The method for analyzing nitrogen isotopes in lunar soil according to claim 8, wherein the analysis system further comprises a first vacuum pump and a second vacuum pump; the heating chamber is in communication with an air suction port of the first vacuum pump via a third pipeline, the third pipeline is provided with a fifth valve, and an air outlet of the quadrupole mass spectrometer is in communication with an air suction port of the second vacuum pump via a fourth pipeline.

10. The method for analyzing nitrogen isotopes in lunar soil according to claim 9, wherein each of the first valve, the second valve, the third valve, the fourth valve, the fifth valve and the control valve is an electromagnetic valve.
